(19) United States
(12) Patent Application Publication
(10) Pub. No.: US 2009/0099539 A1
(43) Pub. Date: Apr. 16, 2009

(54) Potty training liners

(76) Inventor: Lori Lee Periman, Cedar Park, TX (US)

Correspondence Address: Lori Periman, 2308 London Ln, Cedar Park, TX, 78613, US

(21) Appl. No.: 12/285,586

(22) Filed: Oct. 9, 2008

Related U.S. Application Data
(60) Provisional application No. 60/998,825, filed on Oct. 12, 2007.

(51) Int. Cl. A61F 13/15; G09B 23/00

(52) U.S. Cl. 604/361; 604/368; 604/378; 604/367; 434/262

(57) ABSTRACT

Potty training liners are a comfortable, child sized, absorbent liner, with a concave center and an expandable, flexible bottom to allow rapid flowing urine collection and retention. The Liner has unique absorbent collapsible side walls with an unobstructed center containing superabsorbent for quick pooling of urine. The liner is adhered to the inside of the child's underwear with a thick adhesive strip on the bottom outer portion of the liner. The Liner is soft and flexible, and does not have the feel of a diaper. It is a unique stop-gap solution for children progressing from pull-on diapers to unprotected underwear. The purpose of the liner is for training, and short term emergency “accident” protection, as opposed to long term wearable protection.

### FIELD OF THE INVENTION

This invention relates generally to devices that function as an aid to children transitioning from diapers to underwear.

### BACKGROUND OF THE INVENTION

Pull-on type diapers are presently used for potty training, as they allow a child to pull them down, like underwear, to go to the bathroom.

### BRIEF DESCRIPTION OF THE DRAWING

The invention may be best understood by reading the disclosure with reference to the drawing, wherein:

FIG. 1 contains a top view of a potty training liner embodiment adapted for use by a girl;

FIG. 2 contains a transverse partial cross-sectional view taken near the rear liner wall of the FIG. 1 embodiment;

FIG. 3 contains a top view of a potty training liner embodiment adapted for use by a boy; and

FIG. 4 contains a top view of a potty training liner embodiment adapted for use overnight; and

FIG. 5 contains a transverse cross-sectional view taken through an embodiment.

### DETAILED DESCRIPTION OF THE EMBODIMENTS

Although pull-on type diapers do not keep children, who can sense that they need to go to the bathroom, from going, it is now believed that pull-on type diapers have characteristics that do not assist the child in progression from the security of a diaper to wearing unprotected underwear. Pull-on type diapers have a bulky “diaper” feel to them, like the traditional diapers a child has worn since birth. Accordingly, the use of pull-on type diapers for potty training can send confusing messages to children.

The present embodiments are designed for use with underwear, as a transition between traditional or pull-on type diapers and underwear alone. These embodiments are referred to herein as “potty training liners,” and consist of disposable inserts for use with underwear. Potty training liners are preferably designed to be used after a child gains some bladder control (e.g., using pull-on type diapers) and is ready to progress to wearing real underwear. Underwear outfitted with potty training liners does not have the “diaper-feel” of pull-on diapers, and yet gives some protection against unimpeded urine flow onto clothing, carpets, and furniture, etc.

Potty training liners enable a child to wear real underwear earlier while reducing the risk of unnecessary embarrassment and clean-up. Most children do not want to soil their clothes and are often embarrassed at their mistake. A child who has some bladder control knows he “has to go,” but simply waits too long, or gets distracted and forgets he has to go. This typically results in a child not quite making it to the bathroom in time. Potty training liners can save a child in this situation from soiling their clothes, and other items, and the bulky feel of a saturated liner creates awareness prompting the child to take action earlier and allowing them to learn.

Potty training liners are designed for preferable use as “short-term” emergency protection, whereas diapers and sanitary pads are designed for “long-term” wearable protection. At least some preferred embodiments of potty training liners are not designed to keep wetness away from the child, or to allow a child to continue playing after having an accident, but simply keep wetness confined to the pad until they can get to a bathroom or get assistance.

It is now believed that the use of potty training liners will speed the process of potty training by getting children into real underwear much faster, and creating greater awareness of the child's need to go, when they first realize they should, without the added negative consequences of having an unprotected accident. Many busy parents keep children in diapers or pull-on diapers longer, because they fear the clean-up and hassle. Often, parents do not have the time to watch their child as closely as is required when transitioning to real underwear, and frequently forget to remind their child to go to the bathroom. Additionally, liners are preferable to pull-on diapers because they are easier to change. While the pull-on diapers have easy-tear sides for quick removal, the child must still fully undress to put on a clean dry pull-on diaper. Potty training liners will save time by cutting down on clothing changes and redressing. Ultimately, potty training liners help the child “self-train” and keep the experience positive. 191 Sanitary and incontinence pads currently exist in the marketplace for use by adults, but are not believed to meet the needs of a child learning bladder control. Perhaps most important, neither sanitary nor incontinence adult pads will fit or function properly for a child. Sanitary pads are developed to absorb slower, thicker body fluids and hold them over long periods of time. Likewise, incontinence pads are designed for incontinent adults, who typically produce small urinary leaks over time. Children in the potty training process typically have the bladder control to hold their urine until their bladder is nearly full, followed by an urgent release that produces a high volume of urine in a brief time period. Adult incontinence pads are far too large and bulky for a child. In addition, adult incontinence pads are not designed to aid the potty training process because they do not have a dry lightweight feel required for training and differentiating the bulky diaper-like feel between a child's legs.

The preferred embodiments do not protect the child from wetness, as this is not conducive to potty training, nor do they provide long-term wet wearability, like a diaper or other pad. The preferred embodiments do, however, contain several features designed to enhance the comfort and wearability of the dry liner. In preferred embodiments, training liners do not have the feel of a diaper or pull-on training-pant, which has the feel of a diaper. The size and shape, along with compressible side walls, give a comfortable, flexible fit for a small child while providing substantial protection from unexpected puddles on floors and furniture. The flexible bottom and absorbent side wall design retains larger volumes of urine more quickly, by giving the urine somewhere to go besides over the sides, catching and containing it at a faster rate than existing pads. The liners have a form fitting rise at the middle, and rear, of the liner in attempt to protect overflow along the natural curves of the body, while urine is absorbed. The liners also have adhesive limited to the outer portions of the underside of the liner, so that the center can flex and provide expansion as urine is collected and absorbed. This design works with cloth undergarments which naturally give a little in the center.

It is preferable to progress to training liners over pull-on diapers for several reasons. Currently, pull-on diapers require undressing and redressing to change into a new dry pull-on diaper, and they have the same feel as an infant's diaper. Further, pull-on diapers disperse urine in the pant, providing long term wear-ability. This has the effect of numbing awareness and encouraging a child to continue playing and not address the situation. Children must learn to take action to prevent accidents. Part of this learning process is knowing “when” to act, especially when attention is focused on something exciting, like playing. The preferred liners collect urine more centrally, increasing awareness. As the preferred liner becomes saturated, it feels awkward and heavy between the child's legs, prompting the child to take action. Furthermore, training liners do not require a full undress and redress to change into a new dry liner, making the change faster and easier for parents, and giving kids the ability to self-train.

Most absorbent products on the market are designed with at least three layers consisting of a liquid permeable top layer, an absorbent core, and a non-permeable bottom layer. Absorbent materials used in various absorbent products are known. Typically, such products comprise an absorbent fibrous matrix of cotton or wood pulp fluff enhanced with a high-absorbency material known as “superabsorbent.” Superabsorbents are typically crosslinked polymers capable of absorbing 10-100 times their weight in water. Superabsorbents are frequently used in diapers and personal care products to enhance absorption. There are many ways, known to those in the art, to combine superabsorbent and absorbent materials. For example, U.S. Pat. No. 5,356,403, issued to Faulks, et al., describes a layered fibrous structure laced with varying amounts of superabsorbent material at various densities. Other methods involve weaving the superabsorbent into a single fibrous pad during production, or sandwiching a superabsorbent between two fibrous layers.

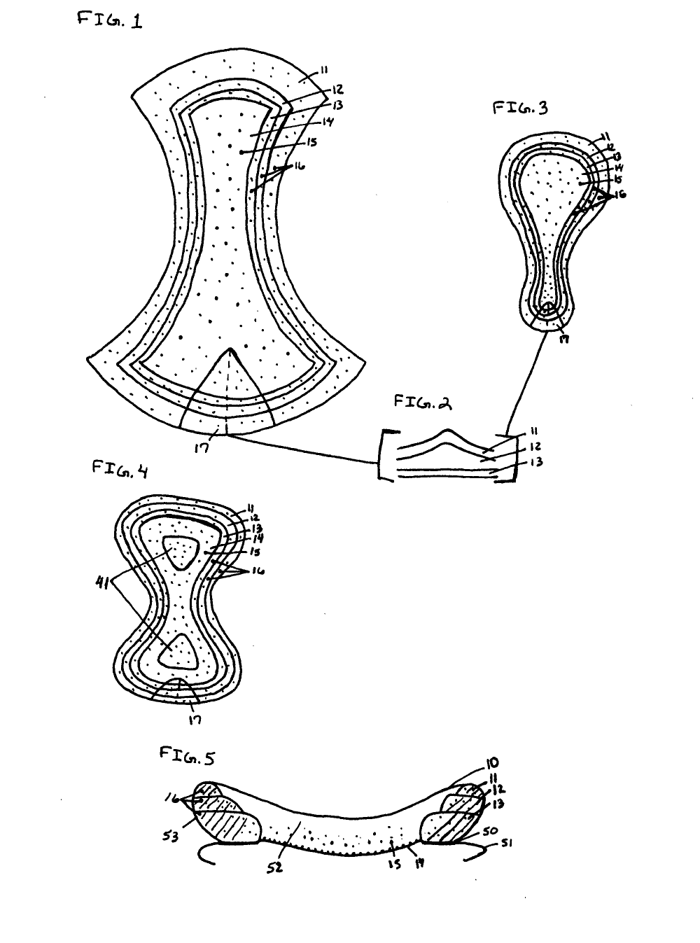

The preferred embodiments are illustrated in FIG. 1 (Top view of liner adapted for girls); FIG. 2 (partial rear transverse cross section); FIG. 3 (Top view of liner adapted for boys); FIG. 4 (Top view of liner adapted for use overnight); and, FIG. 5 (traverse cross-section). The first layer 10, (FIG. 5) comprises a thin, soft, absorbent top sheet, such as absorbent Rayon-polyester with low density, or other non-woven material, which covers and protects the underlayers (the top layer is removed in the drawings FIGS. 1, 2, 3, and 4). Urine moves immediately through this top layer with minimal resistance.

The bottom layer 14 is a soft flexible waterproof material which contains loose superabsorbent material, with or without sparse fibrous material mixed in to aid in preventing gel block. 15. The superabsorbent is typically a hydrophilic polymer made of fine particles of an acrylic acid derivative, such as sodium acrylate, potassium acrylate, or an alkyl acrylate. Further, the flexible bottom layer 14 has a light coating of adhesive on the inside to hold some of the powdered superabsorbent in place and to ensure coverage. The loose superabsorbent 15 allows gravity to dictate where it is needed most. The bottom 14 is designed to flex, this allows side compression for comfort and expansion for increased urine retention. Unlike current absorbent pads, cloth underwear gives and will allow additional expansion in the event of an accident.

The center of the liner 52 is substantially hollow, or concave, so that urine may flow easily into the space while allowing the top layer 10, and bottom layer 14, to sag when the sides are compressed creating space for urine to pool as it is being absorbed. Currently, when sanitary napkins or diapers are compressed on the side, the pad center has a tendency to bunch “up.” This creates overflow, when fluid hits the surface faster than it can be absorbed. Allowing the center to compress downward provides a more comfortable fit, and affords space for urine retention, while avoiding the stiff feel of a diaper or pad.

The center of the liner 52 is surrounded by a soft absorbent wall (11,12,13). The preferred wall configuration contains stacked absorbent and superabsorbent material in a stair-step fashion extending outward. The wall is made of three layers, a bottom layer 13, a middle layer 12 set off-center toward the outside, and a top layer 11 set off-center toward the outside. This creates a lower profile, collapsible wall, that minimizes blockage of urine flow to the center of the liner. Each layer contains superabsorbent 16 which causes the wall to swell, further trapping urine. This configuration allows for sides to compress comfortably while absorbing and directing fast streaming urine toward the center of the liner where it can be contained until the superabsorbent has had time to work. Extra absorbent material in the liner mid-section wall 11,12,13 can provide additional protection along the natural curve of the body.

The fibrous absorbent material used in the wall (11,12,13) may be formed from natural or synthetic fibers and by using methods such as air laying, spunbond, meltblown, or any of the methods known to those skilled in the art for making absorbent fibrous materials. The fibrous layer contains a superabsorbent 16. The superabsorbent may be dispersed through the fibrous layer at the time the fibrous layer is created, or sandwiched between fibrous layers, or by any means available to those skilled in the art.

Additionally, the top two layers in the wall of the rear area 17 may have extra absorbent material, creating a form-fitting rise to help catch potential overflow to the rear when sitting or lying down.

Liners may be designed as unisex, but for comfort, and a lower profile, the preferred liners will be designed based upon gender specific needs. The boy's liner, FIG. 3, will be a somewhat relaxed “sport-cup” shape, with a larger catch area in the front, and an extended reserve in the rear area.

Liners for Girls, FIG. 1, will be a more oblong hourglass shape, with a larger catch area in the rear, and a larger reserve area in the front, but will not extend as far in the front.

Liners designed for overnight protection, FIG. 4, will be longer, as well as contain extra absorbent material in the walls (11,12,13), and in the front and rear center 41 sections, to help catch urine while a child is lying down.

Liners designed for advanced trainers, who are almost finished with potty-training, will have thinner walls and a lower profile, to catch the little leaks that escape when they are trying to get to the bathroom.

Colors can vary, and may include fun child like designs, and/or a disappearing smiley, or positive reinforcement symbol on the inside top layer of the pad, as a positive indication that the pad is dry. (Ink could disappear, or change to “try again” or “oops” type symbol when wet.)

One of ordinary skill in the art will recognize that the concepts taught herein can be tailored to a particular application in many other advantageous ways.

Although the specification may refer to “an”, “one”, “another”, or “some” embodiment(s) in several locations, this does not necessarily mean that each such reference is to the same embodiment(s), or that the feature only applies to a single embodiment.

FIG 1. Top Down View of Girl's Liner with Top Sheet Removed:

11. Top layer of absorbent outer wall containing superabsorbent

12. Second layer of absorbent outer wall containing superabsorbent

13. Third layer of absorbent outer wall containing superabsorbent

14. Flexible waterproof bottom layer, coated with light dusting of adhesive and superabsorbent

15. Loose superabsorbent, or loose superabsorbent mixture with a fibrous material

16. Superabsorbent disbursed through side walls

17. Absorbent material added to rear wall to form an inverted V for added leak protection

### FIG 2. Exploded Horizontal View of the Rear Liner Wall:

11. Rear overflow protection. Top layer of absorbent wall with additional absorbent material forming a protective peak in rear

12. Rear overflow protection. Second layer of absorbent wall with additional absorbent material forming a protective peak in rear

13. Third layer of absorbent wall with superabsorbent material

FIG 3. Top Down View of Overnight Liner with Top Sheet Removed:

11. Top layer of absorbent outer wall containing superabsorbent

12. Second layer of absorbent outer wall containing superabsorbent

13. Third layer of absorbent outer wall containing superabsorbent

14. Flexible waterproof bottom layer, coated with light dusting of adhesive and superabsorbent

15. Loose superabsorbent, or loose superabsorbent mixture with a fibrous material

16. Superabsorbent disbursed through side walls

17. Absorbent material added to rear wall to form an inverted V for added leak protection

FIG 4. Top Down View of Overnight Liner with Top Sheet Removed:

11. Top layer of absorbent outer wall containing superabsorbent

12. Second layer of absorbent outer wall containing superabsorbent

13. Third layer of absorbent outer wall containing superabsorbent

14. Flexible waterproof bottom layer, coated with light dusting of adhesive and superabsorbent

15. Loose superabsorbent, or loose superabsorbent mixture with a fibrous material

16. Superabsorbent disbursed through side walls

17. Absorbent material added to rear wall to form an inverted V for added leak protection

41. Extra absorbent material containing superabsorbent added to front and rear center

### FIG 5. Horizontal End Cut Away View:

10. Low density, liquid permeable top sheet

11. Top layer of absorbent outer wall containing superabsorbent

12. Second layer of absorbent outer wall containing superabsorbent

13. Third layer of absorbent outer wall containing superabsorbent

14. Flexible waterproof liner bottom, coated inside with adhesive and superabsorbent

15. Free floating superabsorbent, or superabsorbent mixture with a fibrous material

16. Superabsorbent located inside wall material

50. Adhesive applied to bottom of flexible liner, under wall, to attach liner to underwear

51. Adhesive flap to wrap around underwear seam and secure liner to underwear

52. Substantially hollow, concave center

53. Waterproof liner extended over side wall to keep clothes dry

1. A child's undergarment liner for urine collection, comprising:
(a) An absorbent, water permeable top layer, whereby body fluid is quickly absorbed.
(b) Absorbent material core, whereby containing urine.
(c) Waterproof bottom layer, whereby preventing leaks and protecting clothing.
(d) Said liner having a predetermined, child specific size and shape, whereby the unique urine collection needs of a child is incorporated.
(e) Containing a superabsorbent, whereby urine is absorbed.
(f) Means for joining said materials.
(g) Means for connecting said materials to undergarment.

2. The absorbent, water permeable, top layer of claim 1 wherein said top layer is formed of a low density material.

3. The absorbent, water permeable, top layer of claim 2 wherein said low density material is of a soft material.

4. The top layer of claim 1, further includes a positive reinforcement mark, which fades when wet.

5. The absorbent material core of claim 1, further contains a superabsorbent, whereby increasing absorption capacity.

6. The absorbent material core of claim 1, further includes a depressed center, whereby creating an absorbent wall with a concave center, wherein urine is quickly pooled and contained.

7. The absorbent wall of claim 6 wherein said absorbent wall is comprised of stair-stepped layers, whereby creating a wide, but flexible catch area.

8. The absorbent wall of claim 6, further includes waterproof outer edge, whereby protecting clothing and containing leaks.

9. The absorbent material core of claim 6, further contains a superabsorbent, whereby the walls expand helping to prevent overflow.

10. The absorbent wall of claim 6, further includes additional absorbent material in the shape of an inverted V, in the rear section of the wall, whereby preventing leaks to the rear when sitting or lying down.

11. The hollow center of claim 6, further includes loose superabsorbent, whereby allowing gravity to dictate where it is needed most.

12. The waterproof bottom layer in claim 1, wherein the bottom layer is an expandable material, whereby increasing holding capacity.

13. The waterproof bottom layer in claim 12, further includes a layer of superabsorbent, whereby fluid is gelled.

14. The waterproof bottom layer in claim 12, further includes loose superabsorbent.

15. The waterproof bottom layer in claim 14, further includes an adhesive applied to the upward-facing inside surface and contains loose superabsorbent, whereby the adhesive ensures some broad dispersion of superabsorbent.

16. The means for connecting said materials to undergarments in claim 1, wherein adhesive is applied to the garment facing side of the liner.

17. The adhesive applied to the underside of the waterproof bottom in claim 16, wherein is limited to the outside portion of the underside of the bottom, whereby allowing the liner to be secured to the undergarment seams, and take advantage of the cloth undergarments ability to give at the center.

18. The means for connecting said materials to undergarments in claim 16, further includes an adhesive side flap to connect the outer edges of the liner to the underside of the undergarment seam.

19. The predetermined size and shape in claim 1, wherein the size and shape will be determined by gender specific requirements, whereby, boys will be shaped substantially like an athletic-supporter, girls will be shaped substantially like an hourglass.

20. A method of containing body fluid, comprising:
(a) an absorbent undergarment liner having a predetermined shape, with a low density absorbent top sheet, a flexible bottom, absorbent side walls, and a concave or hollow core containing superabsorbent.
(b) inserting said liner into the undergarment crotch area, whereby the said side walls will be compressed by the inner thigh.
(c) causing said side walls to create a primary flow channel.
(d) causing said top sheet to create a secondary recessed center channel.
(e) directing body fluid through and over said primary and secondary channels, to the ends of said liner and bottom of said liner.
(d) causing said fluid, not absorbed by said side wall, to fall through widening channel ends to the substantially hollow center below.
(e) gelling said fluid, whereby containing the fluid inside the center.

21. A method of potty training, comprising:
(a) an absorbent undergarment liner having a predetermined child size and shape, with an absorbent top sheet, absorbent core, superabsorbent, and a waterproof bottom with means to attach to undergarment.
(b) inserting said liner into the undergarment crotch area.
(c) catching urine accidents in said liner.
(d) gelling said urine with expanding superabsorbent.
(d) causing said gelled urine to feel heavy, awkward, and uncomfortable in cloth undergarments.
(e) causing said child to seek out assistance or change liners, whereby self training.
